(19) United States
(12) Patent Application Publication
(10) Pub. No.: US 2005/0107612 A1
(43) Pub. Date: May 19, 2005

(54) Process for preparation of montelukast and its salts

(75) Inventors: Buchi Reddy Reguri, Hyderabad (IN); Satyanarayana Bollikonda, Hyderabad (IN); Veera Venkata Naga Chandra Sekhar Bulusu, Hyderabad (IN); Ravi Kumar Kasturi, Hyderabad (IN); Sanjeev Kumar Aavula, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LIMITED; DR. REDDY'S LABORATORIES, INC.

(21) Appl. No.: 10/748,865

(22) Filed: Dec. 30, 2003

(30) Foreign Application Priority Data
Dec. 30, 2002 (IN) 993/MAS/2002

(51) Int. Cl. C07D 215/16

(52) U.S. Cl. 546/177

(57) ABSTRACT

A process for preparation of montelukast or a salt thereof is provided, the process including reacting a late intermediate compound which is 2-[1-[1-R-3-[2-(7 chloro quinolin-2-yl) vinyl [phenyl]-3-[2-methoxy carbonyl phenyl] propyl sulfonyl methyl] cyclo propyl] acetic acid or a salt thereof with methyl magnesium chloride or methyl magnesium bromide in an organic solvent. In one embodiment, the process includes reacting an earlier intermediate compound which is methyl 2-(3-R-(3-(2-(7-chloro 2-quinolinyl)-ethenyl)-3 hydroxy propyl) benzoate with methane sulfonyl chloride or toluene sulfonyl chloride to obtain a mesylated or tosylated derivative of the earlier intermediate compound; followed by a reaction of the mesylated or tosylated derivative with 1-mercapto methyl cyclopropane acetic acid in a polar solvent in a presence of a base to obtain the late intermediate compound.

### CROSS-REFERENCE TO RELATED APPLICATIONS

This application claims the benefit of priority of Indian Patent Application Nos. 993/MAS/2003, filed Dec. 30, 2003, the disclosure of which is incorporated herein by reference in their entirety.

### BACKGROUND OF THE INVENTION

The sodium salt of montelukast is a leukotriene antagonist. It is useful in treatment of asthma, inflammation, allergies, angina, cerebral spasm, glomerular nephritis, hepatitis, endotoxemia, uveitis and allograft rejection.

Certain processes for preparation of montelukast and its salts are known. For example, European Patent No. 480717 discloses certain substituted quinoline compounds, including sodium salt of montelukast, methods for their preparation, and methods of pharmaceutical formulations using these compounds. The process disclosed in EP 480717 includes reacting 2-(2-(2-(3(S)-(3-(2-(7-chloro-2-quinolinyl)-ethenyl) phenyl)-3-(methanesulfonyloxy) propyl) phenyl)-2-propoxy) tetra hydro pyran with methyl 1-(acetylthiomethyl) cyclopropane acetate in presence of hydrazine, cesium carbonate in acetonitrile as solvent to get the methyl ester of montelukast in pyran protected form. The protected compound is further reacted with pyridinium p-toluene sulfonate, sodium hydroxide in a mixture of methanol and tetrahydrofuran as a solvent to afford montelukast sodium.

U.S. Pat. No. 5,614,632 discloses a process for the preparation of the sodium salt of montelukast and certain process intermediates. The process involves generation of dilithium dianion of 1-(mercaptomethyl) cyclopropaneacetic acid followed by condensation with 2-(2-(3(S)-(3-(2-(7-chloro-2-quinolinyl) ethenyl) phenyl)-3-methanesulfonyloxypropyl) phenyl)-2-propanol (referred as mesylated alcohol) to afford montelukast, which is further converted to the corresponding sodium salt via dicyclohexyl amine salt. The '362 patent also discloses a process for the preparation of crystalline montelukast sodium salt and mesylated alcohol. The process involves reacting methyl 2(3 (S)-(3-(2-(7-chloro-2-quinolinyl) ethenyl) phenyl)-3-hydroxy propyl) benzoate with methyl magnesium chloride to give a diol, which is further converted to mesylated alcohol on reaction with methane sulfonyl chloride.

While certain processes of its preparation are known, there is a continuing need for new processes of preparation of montelukast and its salts.

### SUMMARY OF THE INVENTION

In accordance with one aspect, the invention provides a process for preparation of montelukast or a salt thereof that includes reacting a late intermediate compound which is 2-[1-[1-R-3-[2-(7 chloro Quinolin-2-yl) vinyl [phenyl]-3-[2-methoxy carbonyl phenyl] propyl sulfonyl methyl] cyclo propyl] acetic acid or a salt thereof with methyl magnesium chloride or methyl magnesium bromide in an organic solvent. In one preferred embodiment, the process further includes reacting an earlier intermediate compound which is methyl 2-(3-R-(3-(2-(7-chloro 2-quinolinyl)-ethenyl)-3 hydroxy propyl) benzoate with methane sulfonyl chloride or toluene sulfonyl chloride to obtain a mesylated or tosylated derivative of the earlier intermediate compound; followed by a reaction of the mesylated or tosylated derivative with 1-mercapto methyl cyclopropane acetic acid in a polar solvent in a presence of a base to obtain the late intermediate compound. Various additional and/or alternative steps and/or processes are also provided. Each of such processes and steps is contemplated as a separate invention.

In accordance with another aspect, the invention provides a process for preparation of montelukast sodium that includes i) providing a solution of starting montelukast free acid in a halogenated solvent, aromatic solvent, or mixtures thereof; ii) treating the solution with an alcoholic base to convert montelukast free acid into a sodium salt of montelukast; and iii) adding a cyclic or acyclic hydrocarbon solvent to precipitate the sodium salt of montelukast. In one preferred embodiment, the starting montelukast free acid is generated in situ from an amine salt of montelukast in the presence of an organic acid, preferably, acetic acid. Various additional and/or alternative steps and/or processes are also provided. Each of such processes and steps is contemplated as a separate invention.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

For purposes of the present invention, the following terms are defined below.

“Pharmaceutically acceptable” means that which is useful in preparing a pharmaceutical composition that is generally non-toxic and is not biologically undesirable and includes that which is acceptable for veterinary use and/or human pharmaceutical use. A “compound” is a chemical substance that includes molecules of the same chemical structure. When referring to a chemical reaction, the terms “treating”, “contacting” and “reacting” are used interchangeably herein and refer to adding or mixing two or more reagents under appropriate conditions to produce the indicated and/or desired product. It should be appreciated that the reaction which produces the indicated and/or desired product may not necessarily result directly from the combination of two reagents which were initially added, i.e., there may be one or more intermediates which are produced in the mixture which ultimately leads to the formation of the indicated and/or the desired product.

Montelukast, which is also known as [R-(E)-1-[[[1-[3-[2-[7-chloro-2-quinolinyl]ethenyl]phenyl]-3-[2-(1-hydroxy-1-methylethyl)phenyl]propyl]thio] methyl]cyclopropane acetic acid, is the compound of the structure:

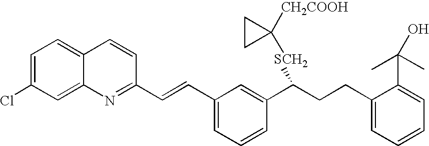

The invention provides a novel route to montelukast and its salts. In general term, the processes of the invention are set forth in the claims. Each step of the process is separately contemplated. For the purpose of illustration, the chemical transformations in accordance with one particular embodiment of the process of the invention may be depicted as follows:

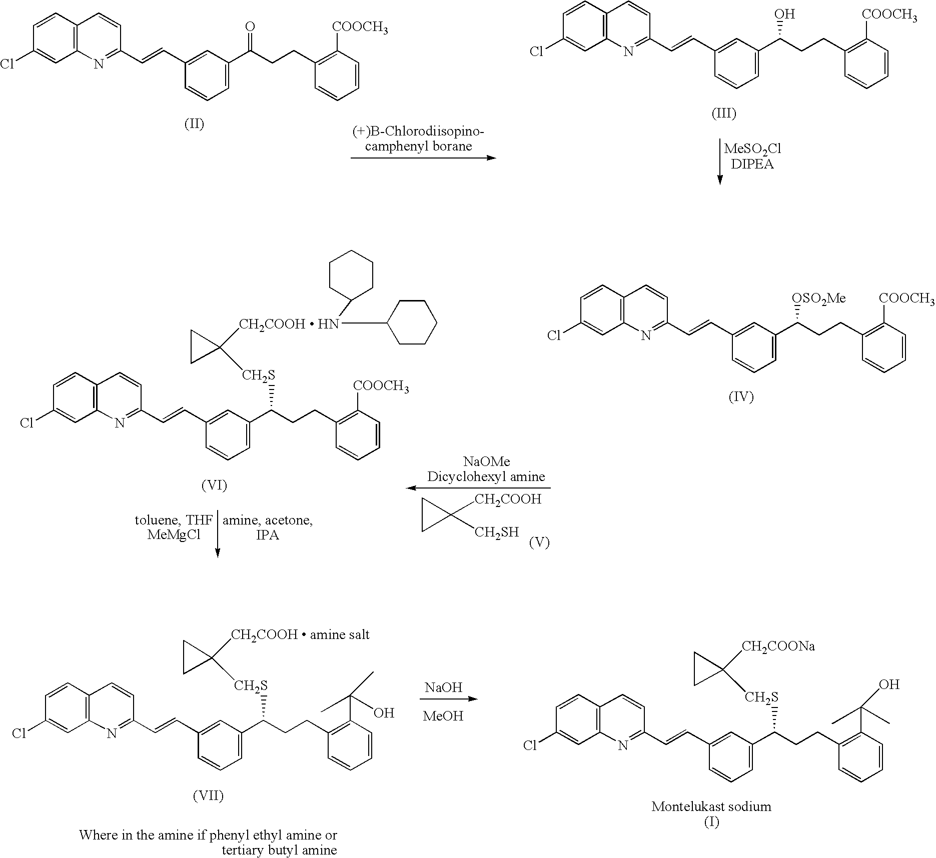

In the illustrated embodiment, initially, methyl-2-(3-(3-(2-(7-chloro-2-quinolinyl)ethenyl)phenyl)-3-oxopropyl benzoate of Formula (II) is reduced with (+) B-chloro diisopinocampheylborane as a chiral reducing reagent in polar organic solvents to result methyl-2-(3-(3-(2-(7-chloro-2-quinolinyl)ethenyl)phenyl)-3-hydroxy propyl benzoate of Formula (III). The compound of formula (III) is mesylated with methane sulfonyl chloride or tosylated with toluene sulfonyl chloride to form methyl-2-(3-(3-(2-(7-chloro-2-quinolinyl)ethenyl)phenyl)-3-methane sulfonyloxy propyl benzoate of formula (IV) or a corresponding tosylate. Also, use of other leaving group-containing compounds instead of the mesylate or tosylate intermediate is also contemplated. The reaction may process in polar solvent or non-polar solvent, preferably, in a mixture of polar and non-polar organic solvents. The resulting leaving group containing intermediate (e.g., mesylate or tosylatye, preferably, mesylate (IV)) is then condensed with 1-mercapto methyl cyclopropane acetic acid of formula (V) in the presence of a base. The use of a mixture of polar organic solvents is preferred. The product of this reaction is preferably isolated in the form of an organic amine salt of formula (VI), preferably, dicyclohexyl amine salt. The resultant amine salt is reacted with methyl magnesium chloride or methyl magnesium bromide in an organic solvent to get montelukast free acid and is again converted to its organic amine salt of formula (VII) to get more pure compound. The amine salt of montelukast of formula (VII) is conveniently converted into pharmaceutically acceptable salts, preferably sodium salt using sodium methoxide or sodium hydroxide.

In a specific embodiment, the invention provides a process for the preparation of montelukast and its pharmaceutically acceptable salts, preferably, its sodium salt, which involves:
a) adding (+) B-chloro diisopinocampheylborane (a chiral reducing agent) to a haloalkane solvent; for example, dichloromethane, dichloroethane or chloroform, preferably, dichloromethane, or an ethereal solvent, for example, as tetrahydrofuran under nitrogen atmosphere at a temperature of −25 to +20° C., preferably-5 to 10° C.;
b) adding a solution of methyl-2-(3-(3-(2-(7-chloro-2-quinolinyl) ethenyl) phenyl)-3-oxopropyl benzoate of Formula (II) in an organic solvent, for example, one of the those described as in step (a), at a temperature of −25 to +20° C., preferably −5 to 10° C., followed by stirring the mass till the reaction substantially completes;
c) quenching the mass with an organic or inorganic base and subsequently working up the mixture to obtain a hydroxy compound of formula (III);
d) reacting the hydroxyl compound obtained in step (c) with methane sulfonyl chloride in the presence of a tertiary amine, for example, diisopropyl ethyl amine or triethyl amine, in a polar and non-polar or mixture of organic solvents at a temperature of 0-75° C.;
e) stirring the reaction mass obtained in step (d) till the reaction substantially completes and subsequently working up the mixture to obtain the mesylated compound of formula (IV);
f) reacting the mesylated compound of formula (IV) with 1-mercapto methyl cyclopropane acetic acid of formula (V) in polar organic solvents or mixture of polar solvents in the presence of a base, for example, sodium methoxide, sodium ethoxide, sodium hydride or n-butyl lithium at a temperature of 30-90° C.;
g) stirring the reaction mass obtained in step (f) till the reaction substantially completes and subsequently working it up to obtain a crude ester compound, which is then reacted with dicyclohexyl amine salt to afford the amine salt of formula (VI);
h) treating the amine salt of formula (VI) with an organic acid, preferably, acetic acid, and further reacting the mass with methyl magnesium chloride or methyl magnesium bromide in an organic solvent, for example, tetrahydrofuran, diethyl ether, diisopropyl ether, 2-methoxy ethanol, toluene, ethyl benzene, 1,4-dioxan or mixtures thereof at a temperature of −10 to +50° C.;
i) stirring the reaction mass obtained in step (h) till the reaction substantially completes and subsequently working it up to isolate montelukast acid from solvents, such as toluene, ethyl acetate, acetonitrile, heptane and hexanes;
followed by a purification of the free acid compound from a solvent selected from polar solvents such as toluene, methanol, ethanol, isopropanol, n-propanol, ethyl acetate, methyl acetate, acetonitrile or mixtures thereof; and
further reacting the resultant montelukast free acid with primary, secondary or tertiary amines, preferably tertiary butyl amine or phenyl ethyl amine to afford the amine salt of formula (VII);
j) purifying the amine compound obtained in step (i) from an organic solvent selected from toluene, methanol, ethanol, isopropanol, n-propanol, ethyl acetate, methyl acetate, acetonitrile or mixtures thereof;
k) converting the amine salt into its pharmaceutically acceptable salts by generating the montelukast free acid from montelukast amine salt in halogenated solvents, for example, chloroform, dichloromethane or dichloroethane, preferably, dichloromethane, or an aromatic hydrocarbon, for example, toluene, ethyl benzene or xylene, preferably, toluene, or mixtures thereof, in the presence of an organic acid, preferably, acetic acid;
l) distilling the solvent of step (k) under reduced pressure at below 60° C. to obtain a residue;
m) dissolving the free acid of step (l) in a halogenated solvent, such as chloroform, dichloromethane or dichloroethane, preferably, dichloromethane, or an aromatic hydrocarbon, such as toluene, ethyl benzene or xylene, preferably, toluene;
n) converting the compound obtained in step (m) to sodium salt of montelukast using sodium hydroxide, sodium methoxide or sodium ethoxide in alcohols such as from methanol, ethanol, propanol, butanol, 2-propanol or tert.butanol, preferably, by using methanolic sodium hydroxide;
o) distilling solvent from the reaction solution of step (n) under reduced pressure and dissolving the residue in toluene, ethylbenzene, or dichloromethane;
p) isolating the desired product from step (o) by adding cyclohexane, n-heptane or hexanes;
q) drying the isolated solid at 50-80° C. under vacuum.

The processes described herein have certain advantages over the known processes. For example, the known procedures may include protection and later deprotection of diol intermediate and the use of certain undesirable raw materials, such as n-butyl lithium, in also undesirable reaction conditions (for example, certain art process require commercially undesirable temperatures (e.g., −25° C.)). Likewise, certain processes that are known in the art involve tedious workup to isolate the required product resulting in excess time cycle. The process of the present invention is cost effective, eco-friendly and well suited for scale up. The montelukast sodium prepared by the processes described herein is suitable for pharmaceutical formulations. The montelukast sodium obtained in the present novel process is having >99.0% enantiomeric excess purity and resulted in amorphous form. The montelukast sodium obtained in the present process is also free flowing and non-solvated solid; hence it is well suited for pharmaceutical applications.

The invention is further defined by reference to the following examples describing in detail the preparation of the compound and the compositions of the present invention, as well as their utility. It will be apparent to those skilled in the art, that many modifications, both to materials, and methods, may be practiced without departing from the purpose and interest of this invention.

### EXAMPLE 1

### Preparation of Methyl 2-(3-R-(3-(2-(7-chloro 2-quinolinyl)-ethenyl)-3 Hydroxy Propyl) Benzoate

398 milliliters (mls) of (+) B-chlorodiisopino camphenyl borane and 1000 mls of dichloromethane were charged into a round-bottomed flask under nitrogen atmosphere. 200 grams of methyl-2-(3-(3-(2-(7-chloro-2-quinolinyl ethenyl) phenyl)-3-oxo propyl benzoate were separately dissolved in 1000 mls of dichloromethane at 25-35° C., and added to the chiral borane reagent slowly at 0 to 5° C. under nitrogen atmosphere. The reaction mass was maintained at 0-5° C. until reaction completion. 130 mls of aqueous ammonia (25% W/V) were charged into the reaction mass with stirring; cooling was discontinued and the mass was stirred at 25-35° C. for 1-2 hours. 20% solution of vacuum salt was added to the reaction mass with stirring, which was continued for another 15-30 minutes.

The organic and aqueous layers were separated, and the organic layer was washed with 3×200 mls of the 20% solution of the vacuum salt. The solvent was removed from the organic layer at an atmospheric pressure and a temperature of below 55° C. The residual solvent was further removed under reduced pressure. The obtained crude solid was re-dissolved in methanol (400 mls). The solvent was again removed under reduced pressure at below 55° C. The crude solid was again re-dissolved in methanol (2400 ml) at 25-35° C., and stirred at 25-35° C. for another 1-2 hours. The undissolved gum material was filtered and the gum was washed with additional methanol (200 mls).

The filtrate was transferred into a clean round bottomed flask. 600 mls of water were added dropwise with stirring during 2-3 hours to precipitate the title compound. The stirring was continued for another 1-2 hours. The solid was filtered and washed with a mixture of methanol (100 mls) and water (100 mls); followed by a final wash with hexanes (400 mls). The resulting solid was dried at 50-60° C. to afford 142 grams of the title compound.

### EXAMPLE 2

### Purification of Methyl 2-(3-R-(3-(2-(7-chloro 2-quinolinyl)-ethenyl)-3 Hydroxy Propyl) Benzoate

142 grams of methyl 2-(3-R-(3-(2-(7-chloro 2-quinolinyl)-ethenyl)-3 hydroxy propyl) benzoate were dissolved in 1704 ml methanol and the mixture was stirred at 25-35° C. for 1 hour. The undissolved gum material was filtered and the gum was washed with methanol (142 ml). The combined filtrates were transferred into a new round-bottomed flask. 426 ml of water were added dropwise at 25-35° C. during 2-3 hours to precipitate the desired compound; stirring continued for further 1-2 hours at 25-35° C. The solid was filtered and washed with a mixture of methanol (71 mls) and water (71 mls); followed by a final wash with hexanes (284 mls). The washed solid was dried at 50-60° C. to afford 103.1 grams of the purified compound.

### EXAMPLE 3

### Preparation of dicyclohexyl amine salt of 2-[1-[1-R-3-[2-(7 chloro quinolin-2-yl) vinyl [phenyl]-3-[2-methoxy carbonyl phenyl] propyl sulfonyl methyl] cyclo propyl] acetic acid

100 grams of methyl 2-(3 R-(3-(2-(7-Chloro 2-Quinolinyl)-ethenyl phenyl)-3-hydroxy prepyl) benzoate and 500 mls of toluene were charged into a round bottomed flask and stirred. The mixture was heated to reflux and maintained at reflux for 1 hour. Approximately 300 mls of toluene were distilled off at atmospheric pressure to remove an azeothrope, and the mass was cooled to 50° C. The remaining solvent was removed under reduced pressure. The residue was re-dissolved in 200 mls of dichloromethane at 25-35° C. The solvent was removed again under reduced pressure. The residue was again re-dissolved in 1000 mls of dichloromethane and the mixture was cooled to 0-5° C.

305 mls of diisopropyl ethylamine (DIPEA) were added at once to the stirred mixture; and the reaction mass was stirred at 0-5° C. for 15-30 minutes. 84.6 mls of methane sulfonyl chloride were added dropwise at 0-5° C. with stirring. After the addition was completed, the cooling was discontinued, and the reaction mass was maintained at 25-35° C. until reaction completion. 600 mls of water were added and the mass was stirred for another 30 minutes.

The organic and aqueous layers were separated, and the aqueous layer was extracted with 200 mls of dichloromethane. The combined organic layers were washed with water (3×600 ml). Dichloromethane was distilled off atmospherically, followed by distillation under reduced pressure at a temperature of below 50° C. The resulting residue was re-dissolved in toluene (200 mls), which was again distilled off under reduced pressure and 45-50° C. to obtain a residue of the mesylate intermediate compound.

47.9 grams of mercapto methyl cyclopropyl acetic acid and 450 mls of methanol were stirred until clear dissolution at 25-35° C. for 60 minutes. A mixture of the crude intermediate mesylate obtained as described above, dichloromethane and dimethyl formamide (450 ml) were added, and the resulting reaction mass was stirred for clear dissolution at 25-35° C. The reaction mass was heated and maintained at reflux temperature for 2-3 hours. 450 mls of water were charged to the reaction mass; stirring continued for 15 minutes. The organic and aqueous layers were separated; the aqueous layer extracted with 200 ml of dichloromethane. The combined organic layers were washed with a mixture of vacuum salt (37.5 grams) and water (400 ml) solution, then washed with a solution of acetic acid (45 ml) in water (400 ml), followed by a water wash (4×400 ml).

The solvents were distilled off under atmospherically from the organic layer; followed by distillation under reduced pressure at 45-50° C. The residue was dissolved in 200 ml acetone; and acetone was re-distilled off under reduced pressure at 45-50° C. Thus obtained residual crude product was re-dissolved in 500 ml acetone at 25-35° C. 51.6 mls of dicyclohexyl amine were added to the solution of the crude residue at 25-35° C.; and the mass was stirred at 25-35° C. until a solid separated. The separated solid was filtered, and the wet cake was taken into 400 ml of acetone, and heated to reflux. The mass was maintained at reflux for 1-2 hours, then cooled to 25-35° C.; stirring continued for 4-5 hours. The resulting solid was filtered and washed with 50 mls of acetone. The solid was dried in an oven at 45-50° C. to afford the 49.7 grams of the title compound.

### EXAMPLE 4

### Purification of Dicyclo Hexyl Amine Salt of 2-[1-[1-(R)-3-[2-(E)-(7chloro Quinolin-2-yl)vinyl[phenyl]-3-[2-methoxy Carbonyl Phenyl]propyl Sulfonyl Methyl]cyclo Propyl]acetic Acid

49 grams of dicyclo hexyl amine salt of 2-[1-[1-(R)-3-[2-(E)-(7chloro quinolin-2-yl)vinyl[phenyl]-3-[2-methoxy carbonyl phenyl]propyl sulfonyl methyl]cyclo propyl]acetic acid and 490 mls of acetone were charged into a round bottomed flask, and the mixture was heated to reflux. The mass was maintained at reflux for 1-2 hours, cooled to 25-35° C. slowly under stirring, and maintained at 25-35° C. for another 4-5 hours. The separated solid was filtered and washed with acetone (49 ml). Drying in an oven at 50-55° C. afforded 44.7 grams of purified title compound.

### EXAMPLE 5

### [R]-1-[[[1-[3-[2-(7-chloro-2-quinolinyl) ethenyl]phenyl]-3-[2-(1-hydroxy-1-methylethyl)-phenyl]propyl]thio]methyl]cyclopropane Acetic Acid (Montelukast Free Acid)

100 grams of dicyclohexyl amine salt of 2-[1-[1-(R)-3-[2-(E)-(7chloro quinolin-2-yl) vinyl [phenyl]-3-[2-methoxy carbonyl phenyl]propyl sulfonyl methyl]cyclopropyl]acetic acid (compound (VI)) and 1000 mls of toluene were charged to a round bottomed flask, and stirred for about 5 minutes. A mixture of acetic acid (15 mls) and water (500 mls) was added, and the mass was further stirred for another 30 minutes. The organic and aqueous layers were separated; the organic layer was washed with water (3×500 ml) and dried over sodium sulphate. The solvent was removed under reduced pressure at a temperature below 50° C. The resulting crude residue was dissolved in a mixture of toluene (760 mls) and tetrahydrofuran (760 mls); the solution was transferred into a round bottomed flask and cooled to 0° C. under nitrogen atmosphere. 261 mls of 3-molar solution of methyl magnesium chloride in tetrahydrofuran were added dropwise during 2-3 hours at 0-5° C. The reaction mass was maintained at 0-5° C. for 6-7 hours, and cooled to 0° C. A mixture of acetic acid (90 mls) and water (750 mls) was slowly added at below 15° C. for about one hour. The reaction mass was stirred at 25-35° C. for another one hour until clear dissolution. The organic and aqueous layers were separated, and the organic layer was washed with 5% sodium bicarbonate solution (2×750 mls), followed by a water wash (2×750 ml). The organic layer was dried over sodium sulphate. The dried organic layer was heated under reduced pressure to remove the solvent. The residue was treated with additional amount(s) of methyl magnesium chloride followed by work (two to three times) until the starting material disappeared.

The crude product was dissolved in toluene (100 ml) and stirred at 25-35° C. to separate a solid. The separated solid was filtered and washed with toluene (30 mls). The wet solid and toluene (90 mls) were charged into a round-bottomed flask, heated to 90° C., and stirred for 30 minutes until complete dissolution, cooled to 25-35° C., and maintained for 6-10 hours. The solid was filtered and washed with toluene (22 mls). The re-precipitation process was repeated four to five times. The solid was dried to afford about 17.4 grams of the purified title compound.

### EXAMPLE 6

### [R]-1-[[[1-[3-[2-(7-chloro-2-quinolinyl) ethenyl]phenyl]-3-[2-(1-hydroxy-1-methylethyl)-phenyl]propyl]thio]methyl]cyclopropane Acetic Acid Tertiary Butyl Amine Salt (Montelukast Tertiary Butyl Amine Salt)

8.6 grams of [R]-1-[[[1-[3-[2-(7-chloro-2-quinolinyl) ethenyl]phenyl]-3-[2-(1-hydroxy-1-methyl ethyl)-phenyl]propyl]thio]methyl]cyclopropane acetic acid, 155 mls of acetone, and 17 mls of isopropyl alcohol were charged into a round bottomed flask and stirred at 25-35° C. until clear dissolution. Tertiary butyl amine was added and the mass was stirred at 25-35° C. The separated solid was filtered, washed with acetone (20 mls) and dried at 40-50° C. The dried residue was re-precipitated from a mixture of acetone (225 mls) and isopropyl alcohol (25 mls), affording 6 grams of the title compound.

### EXAMPLE 7

### [R]-1-[[[1-[3-[2-(7-chloro-2-quinolinyl) ethenyl]phenyl]-3-[2-(1-hydroxy-1-methyl ethyl)-phenyl]propyl]thio]methyl]cyclopropane Acetic Acid Sodium Salt (Montelukast Sodium Salt)

[R]-1-[[[1-[3-[2-(7-chloro-2-quinolinyl) ethenyl]phenyl]-3-[2-(1-hydroxy-1-methyl ethyl)-phenyl]propyl]thio]methyl]cyclopropane acetic acid tertiary butyl amine salt obtained in Example 6 and dichloromethane (50 mls) were charged into a round-bottomed flask at 25-35° C. A mixture of 0.5 mls of acetic acid and 25 mls of water was added to the mass, and stirred at 25-35° C. for 15 minutes. The organic and aqueous layers were separated; the organic layer was washed with water (4×25 mls) and dried over sodium sulphate. The solvent was removed under reduced pressure at a temperature below 45° C. 10 mls of methanol were added to the residue. The solvent was removed again under reduced pressure at a temperature of below 45° C. A mixture of 0.307 grams of freshly prepared sodium pellets and 50 mls of methanol was added to the residue at 25-35° C. 0.5 grams of carbon were added and the mass was stirred for about 30 minutes at 25-35° C. The carbon was filtered and washed with methanol. The filtrates were combined and the solvent was removed under reduced pressure at a temperature below 45° C. The residue was re-dissolved in toluene (25 mls) and the solvent was removed again under reduced pressure at a temperature below 45° C. The residue was re-dissolved in toluene (5 ml) and added to a pre-filtered n-heptane under nitrogen atmosphere at 25-35° C. The mixture was stirred at 25-35° C. for about 1 hour to form a precipitate, which was filtered and washed with n-heptane (25 ml) under nitrogen atmosphere. The resulting solid was dried at 80° C. to afford 3.2 grams of the title compound.

1. A process for preparation of montelukast or a salt thereof, said process comprising reacting a late intermediate compound which is 2-[1-[1-R-3-[2-(7 chloro quinolin-2-yl) vinyl [phenyl]-3-[2-methoxy carbonyl phenyl]propyl sulfonyl methyl]cyclo propyl]acetic acid or a salt thereof with methyl magnesium chloride or methyl magnesium bromide in an organic solvent.

2. The process of claim 1, further comprising reacting an earlier intermediate compound which is methyl 2-(3-R-(3-(2-(7-chloro 2-quinolinyl)-ethenyl)-3 hydroxy propyl) benzoate with methane sulfonyl chloride or toluene sulfonyl chloride to obtain a mesylated or tosylated derivative of said earlier intermediate compound; followed by a reaction of said mesylated or tosylated derivative with 1-mercapto methyl cyclopropane acetic acid in a polar solvent in a presence of a base to obtain said late intermediate compound.

3. The process of claim 1, wherein said late intermediate compound is an amine salt of 2-[1-[1-R-3-[2-(7 chloro quinolin-2-yl) vinyl [phenyl]-3-[2-methoxy carbonyl phenyl]propyl sulfonyl methyl]cyclo propyl]acetic acid.

4. The process of claim 1, wherein said late intermediate compound is a dicyclohexyl amine salt of 2-[1-[1-R-3-[2-(7 chloro quinolin-2-yl) vinyl [phenyl]-3-[2-methoxy carbonyl phenyl]propyl sulfonyl methyl]cyclo propyl]acetic acid.

5. The process of claim 4, wherein said reacting step further includes treating said dicyclohexyl amine salt of 2-[1-[1-R-3-[2-(7 chloro quinolin-2-yl) vinyl [phenyl]-3-[2-methoxy carbonyl phenyl]propyl sulfonyl methyl]cyclo propyl]acetic acid with an organic acid prior to the reaction with said methyl magnesium chloride or methyl magnesium bromide.

6. The process of claim 5, wherein said organic acid is acetic acid.

7. The process of claim 1, wherein said organic solvent is selected from the group consisting of tetrahydrofuran, diethyl ether, diisopropyl ether, 2-methoxy ethanol, toluene, ethyl benzene, 1,4-dioxane, and the mixtures thereof.

8. The process of claim 1, wherein said reacting step is carried out at a temperature ranging from about −10° C. to about 50° C.

9. The process of claim 4, wherein said reacting step further includes converting said dicyclohexyl amine salt of 2-[1-[1-R-3-[2-(7 chloro quinolin-2-yl) vinyl [phenyl]-3-[2-methoxy carbonyl phenyl]propyl sulfonyl methyl]cyclo propyl]acetic acid to a montelukast free acid, followed by a conversion of said montelukast free acid to an amine salt of montelukast.

10. The process of claim 9, wherein said amine salt of montelukast is tertiary butyl amine salt or phenyl ethylamine salt.

11. The process of claim 9, wherein said montelukast free acid is isolated from a solvent selected from the group consisting of toluene, ethyl acetate, acetonitrile, heptane, hexane and mixtures thereof, and purified by precipitating it from a solvent selected from the group consisting of toluene, methanol, ethanol, isopropanol, n-propanol, ethyl acetate, methyl acetate, acetonitrile and mixtures thereof.

12. The process of claim 9, further comprising converting said amine salt of montelukast to a sodium salt of montelukast.

13. The process of claim 1, wherein said starting compound is reacted with methyl magnesium chloride in a mixture of tetrahydrofurane and toluene.

14. The process of claim 2, wherein said base is selected from sodium methoxide, sodium ethoxide, sodium hydride and n-butyl lithium.

15. The process of claim 14, wherein said polar solvent is selected from the group consisting of methanol, dichloromethane, dimethylformamide and mixtures thereof.

16. A process for preparation of montelukast sodium comprising:
(i) providing a solution of starting montelukast free acid in a halogenated solvent, aromatic solvent, or mixtures thereof;
(ii) treating said solution with an alcoholic base to convert said montelukast free acid into a sodium salt of montelukast;
(iii) adding a cyclic or acyclic hydrocarbon solvent to said solution thereby precipitating said sodium salt of montelukast.

17. The process of claim 16, wherein said starting montelukast free acid is generated in situ from an amine salt of montelukast in the presence of an organic acid.

18. The process of claim 16, wherein said halogenated solvent is selected from the group consisting of chloroform, dichloromethane, and dichloroethane.

19. The process of claim 16, wherein said halogenated solvent is dichloromethane.

20. The process of claim 16, wherein said aromatic solvent is selected from the group consisting of toluene, ethyl benzene or xylene.

21. The process of claim 20, wherein said aromatic solvent is toluene.

22. The process of claim 16, wherein said organic acid is acetic acid.

23. The process of claim 16, wherein alcoholic base is selected from the group consisting of sodium hydroxide, sodium methoxide or sodium ethoxide in methanol, ethanol, propanol, butanol, 2-propanol or tert-butanol.

24. The process of claim 16, wherein alcoholic base is methanolic sodium hydroxide.

25. The process of claim 17, wherein said amine salt of montelukast is tertiary butyl amine salt or phenyl ethylamine salt.

26. The process of claim 16, wherein said hydrocarbon solvent is selected from the group consisting of cyclohexane, hexane, n-heptane and mixtures thereof.

27. The process of claim 17, further comprising reacting 2-[1-[1-R-3-[2-(7 chloro quinolin-2-yl) vinyl [phenyl]-3-[2-methoxy carbonylphenyl]propyl sulfonyl methyl]cyclo propyl]acetic acid or a salt thereof with methyl magnesium bromide or methyl magnesium chloride in toluene, tetrahydrofuran, diethyl ether or diisopropyl ether to obtain said amine salt of montelukast.
